(19) United States
(12) Patent Application Publication
(10) Pub. No.: US 2013/0160774 A1
(43) Pub. Date: Jun. 27, 2013

(54) UNITARY PROPHYLACTIC DEVICE

(71) Applicant: Robert J. SCHUMAN, Simpsonville, SC (US)

(72) Inventor: Robert J. SCHUMAN, Simpsonville, SC (US)

(21) Appl. No.: 13/724,746

(22) Filed: Dec. 21, 2012

Related U.S. Application Data
(60) Provisional application No. 61/578,958, filed on Dec. 22, 2011.

(51) Int. Cl. A61F 6/04

(52) U.S. Cl. CPC A61F 6/04
USPC 128/844; 264/219

(57) ABSTRACT

The claimed subject matter provides a prophylactic device that includes a condom portion or section and an integrated shield. The condom and the shield are combined together such that the shield and the condom are made from the same material and are a single piece/sheet of material. In other words, the condom and the shield are combined to form a single prophylactic device that cannot be separated (e.g., not two separate devices).

### CROSS-REFERENCE TO RELATED APPLICATIONS

This application claims the benefit of U.S. Provisional Application Ser. No. 61/578,958, filed Dec. 22, 2011, and entitled “CONDOM WITH INTEGRATED SHIELD.” The entirety of the aforementioned application is incorporated herein by reference.

### BACKGROUND

Sexual transmitted diseases (STDs) are a serious concern and threat to the well-being of people within society. Although a large number of people abstain from sexual activity to ensure complete immunity from such diseases, this group of people has steadily declined for various reasons. For instance, society-based pressures have overcast a negative light on a person who purposely chooses to abstain from sexual activity. These society-based pressures have grown to a driving force behind a person's sexual activeness, while religion and moral-based pressures have continued to decline.

There are various sexually transmitted diseases (STDs) that threaten a sexual active individual. STDs are also referred to as a sexually transmitted infection (STI) or a venereal disease (VD) and can be generalized as an illness that has a significant probability of transmission between individuals through sexual behavior. For instance, the sexual behavior can be vaginal intercourse, oral sex, and anal sex. By way of example, an STI or an STD can be Chancroid (e.g., Haemophilus ducreyi), Chlamydia (e.g., Chlamydia trachomatis), Granuloma inguinale (e.g., Klebsiella Granulomatis), Gonorrhea (e.g., Neisseria gonorrhoeae), Syphilis (e.g., Treponema Pallidum), Candidiasis (e.g., yeast infection), Viral hepatitis (e.g., Hepatitis B virus), Herpes simplex (e.g., Herpes simplex virus 1, 2) Human Immunodeficiency Virus (HIV), HPV (e.g., Human Papillomavirus), genital warts, Molluscum contagiosum (e.g., molluscum contagiosum virus MCV), Crab louse (e.g., colloquially known as “crabs,” “pubic lice,” or Pthirus pubis), Scabies (e.g., Sarcoptes scabiei), Trichomoniasis (e.g., Trichomonas vaginalis), among others.

If an individual is sexually active (e.g., does not abstain from sexual activity), other techniques exist in order to increase prevention of STIs and STDs. In particular, a condom (e.g., a barrier device worn on a penis of the male) can substantially decrease the risks of contracting or transmitting a sexually transmitted disease when such individual is sexually active. The condom has been used for at least the last 400 years. Since around the 19^{th} century, condoms have been one of the most popular devices or techniques of contraception and STI prevention in the world. Although various minor modifications have been implemented with the condom, the actual condom fundamentals have not changed.

### SUMMARY

The following presents a simplified summary of the innovation in order to provide a basic understanding of some aspects described herein. This summary is not an extensive overview of the claimed subject matter. It is intended to neither identify key or critical elements of the claimed subject matter nor delineate the scope of the subject innovation. Its sole purpose is to present some concepts of the claimed subject matter in a simplified form as a prelude to the more detailed description that is presented later.

The subject innovation relates to a unitary prophylactic device. The device includes a shaft portion, reservoir tip, and a shield portion, wherein the device is made from the same material and are a single piece/sheet of material. In other words, the unitary prophylactic device are combined to form a single device that cannot be separated (e.g., not two separate devices). In an embodiment, the unitary prophylactic device are made from the same material with a single mold during a molding process. Moreover, the shield can include creases or folds to enable the shield to fold or collapse toward or away from a shaft of the device. Within such creases or folds, adhesive material can enable the shield to maintain contact with a wearer once the device are unrolled (e.g., opened, open condition) for use. Additionally, due to the construction and aspects (e.g., creases, folds, etc.) of the shield, the device can be packaged in a compact and familiar fashion for consumers. In other words, the unitary prophylactic device can be packaged in a manner so as to maintain familiarity with consumers in relation to packaging.

The following description and the annexed drawings set forth in detail certain illustrative aspects of the claimed subject matter. These aspects are indicative, however, of but a few of the various ways in which the principles of the innovation may be employed and the claimed subject matter is intended to include all such aspects and their equivalents. Other advantages and novel features of the claimed subject matter will become apparent from the following detailed description of the innovation when considered in conjunction with the drawings.

### BRIEF DESCRIPTION OF THE DRAWINGS

FIG. 1 illustrates a device with a shield in an unrolled position (open condition).

FIG. 2 illustrates a device that includes a shield that collapses upon a shaft.

FIG. 3 illustrates a device with a shield at an intermediate position.

FIG. 4 illustrates a device with a shield in a rolled position (closed condition).

FIG. 5 is a cross-sectional view of a mold for a device with a shield as provided by this subject disclosure.

FIG. 6 is a flow chart diagram of a method of creating a mold for a device with a shield.

FIG. 7 is a flow chart diagram of a method of packaging a device with a shield.

### DETAILED DESCRIPTION

The subject innovation relates at least in part to prophylactic device fabricated from one piece of material. A shield is connected to a shaft of the prophylactic device to provide a prophylactic device that affords additional protection. In particular, the unitary prophylactic device can be constructed with an shield to increase protection from Sexually Transmitted Infections (STIs) and Sexually Transmitted Diseases (STDs). The shield can include creases or folds that allow the shield to fold up onto a shaft portion of the device or collapse down onto a shaft of a penis of the wearer. The shield can fold up or collapse onto the shaft portion to allow the device to be rolled up (e.g., closed, closed condition) for a compact and familiar fashion for consumers. Moreover, an adhesive material can be included within the creases or folds to maintain position of the shield onto a wearer during use. Conventional techniques often include two separate devices, a condom and a shield, to provide such protection. Moreover, conventional techniques that include a condom with a shield are often large in size and are sold in large and non-discrete packaging in comparison to a traditional condom packaging.

The claimed subject matter is described with reference to the drawings, wherein like reference numerals are used to refer to like elements throughout. In the following description, for purposes of explanation, numerous specific details are set forth in order to provide a thorough understanding of the subject innovation. It may be evident, however, that the claimed subject matter may be practiced without these specific details. In other instances, well-known structures and devices are shown in block diagram form in order to facilitate describing the subject innovation.

Features that are described and/or illustrated with respect to one embodiment may be used in the same way or in a similar way in one or more other embodiments and/or in combination with or instead of the features of the other embodiments. These and further aspects and features will be apparent with reference to the following description and attached drawings. In the description and drawings, particular embodiments of the subject innovation have been disclosed in detail as being indicative of some of the ways in which the principles of the subject innovation may be employed, but it is understood that the subject innovation is not limited correspondingly in scope. Rather, the subject innovation includes all changes, modifications and equivalents coming within the scope of the claims appended hereto. The accompanying illustrations are examples of the subject disclosure, but the innovation can appear in various embodiments depending on varying sleeve lengths and personal customizations that are not illustrated here.

Of course, those skilled in the art will recognize many modifications may be made to this configuration without departing from the scope or spirit of the claimed subject matter. Moreover, the word “exemplary” is used herein to mean serving as an example, instance, or illustration. Any aspect or design described herein as “exemplary” is not necessarily to be construed as preferred or advantageous over other aspects or designs. It should be emphasized that the term “comprises” or “comprising” when used in this specification is taken to specify the presence of stated features, integers, steps or components but does not preclude the presence or addition of one or more other features, integers, steps, components or groups thereof.

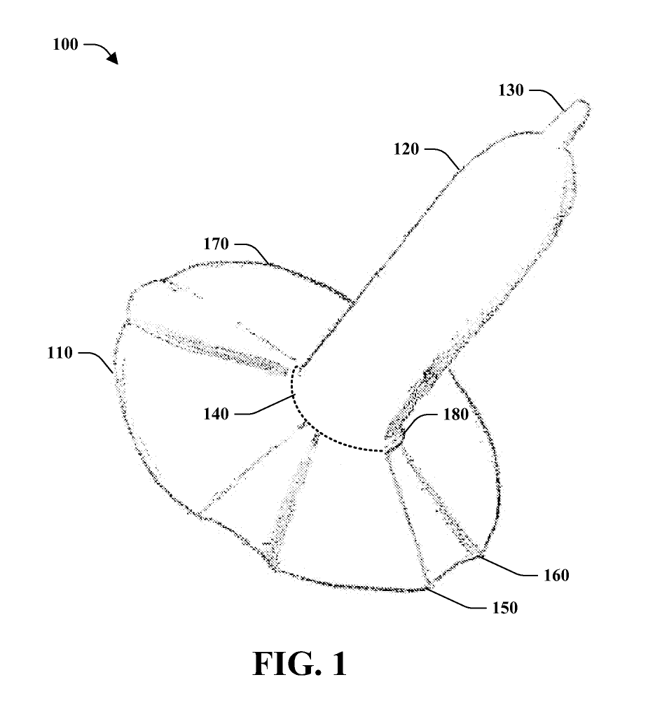

FIG. 1 illustrates a prophylactic device 100 with a shield in an unrolled position (open). The prophylactic device 100 (also referred to as a condom or a device with a condom portion and a shield portion) can include a prophylactic dam 110 (herein referred to as the shield 110). The shield 110 and the prophylactic device 100 are made from a continuous piece of material. In other words, the prophylactic device 100 and the shield 110 are unitary and inseparable. Although the prophylactic device 100 and the shield 110 are discussed as separate elements, the prophylactic device 100 and the shield 110 are integrated to form a single device that protects with a condom section and a shield section. The prophylactic device 100 can include a shaft 120 and a reservoir tip 130 (located at a top portion of the prophylactic device). The shaft 120 can cover a shaft of a penis of the wearer and the reservoir tip 130 can capture semen from the penis of the wearer. At a bottom portion of the prophylactic device 100, an opening is available to allow a penis to be inserted into the prophylactic device 100. Moreover, the bottom portion of the prophylactic device 100 can include transition location 140. The transition location 140 is where the shield 110 meets the shaft 120. The prophylactic device 100 is illustrated in an open condition in which the shield 110 rolled down from the shaft 120 and collapsed away from the shaft 120.

In an embodiment, the shield 110 can be integrated with the prophylactic device 100. For example, the shield 110 can be created and then be integrated into a condom portion during manufacturing, wherein the integration is at the transition location 140. In an embodiment, the shield 110 and the prophylactic device 100 are manufactured together. In either embodiment, the prophylactic device 100 is a unitary device that includes the shield 110, the shaft 120, and the reservoir tip 130.

The prophylactic device 100 can include an inner circumference 180 and an outer circumference 170, wherein the inner circumference 180 is located at the transition location where the shaft transitions to the shield 110. The inner circumference 180 is less than the outer circumference 170. Moreover, the inner circumference 180 is substantially similar to a circumference of the shaft 120. In an embodiment, the device 100 includes a distance between the inner circumference 180 and the outer circumference 170. As the distance increases between the inner circumference 180 and the outer circumference 170, the circumference difference (e.g., outer circumference 170 minus inner circumference 180) increases. Moreover, the transition location 140 can be a pivot point for the shield to collapse onto and toward the shaft 120 or collapse away from the shaft 120. Upon the collapsing motion toward the shaft 120, the one or more creases allow the shield 110 to fold which enables the outer circumference 170 to be reduced (e.g., due to the folds from the creases) to a circumference value approximate to the circumference of the shaft and/or the inner circumference 180.

The prophylactic device 100 include the shield 110 that is frustoconical in shape and includes a top and an opposing based, wherein the top is located at the transition location 140.

The prophylactic device 100 can further include at least two or more creases (e.g., also referred to as a fold or a rib). The shield 110 can include a first crease 150 and a second crease 160. The creases can be pre-fabricated to enable the shield 110 to fold or collapse upward onto the shaft 120. Additionally, the creases enable the shield 110 to fold or collapse downward away from the shaft 120 (e.g., to contact a wearer of the device 100). It is to be appreciated that there can be any suitable number of creases fabricated onto the shield 110 and the number of creases illustrated in FIG. 1 is solely for illustrative purposes only and is not to be limiting on the subject innovation.

The creases (e.g., the first crease 150 and the second crease 160) allow the shield 110 to be folded and/or collapsed upward onto the shaft 120. Alternatively and alternatively, the creases allow the shield 110 to be folded and/or collapsed downward away from the shaft 120. By collapsing or folding the shield 110 upward onto the shaft 120, the prophylactic device 100 with shield 110 can take a more condense shape that takes up less space for packaging, storage, and the like. Such aspect is extremely advantageous in various manners but in particular, in terms of packaging. The prophylactic device 100 with shield 110 can be packaged similar to traditional condoms due to the ability of the shield 110 to collapse and/or fold on the shaft 120. Once collapsed or folded upward, the device 100 and the integrated shield 110 (collapsed, closed, etc.) can be rolled in a space-efficient manner.

In between the first crease 150 and the second crease 160, an area of the shield 110 can include a portion of adhesive. For instance, an adhesive can exist to maintain contact with a portion of the wearer's pubic region, wherein the adhesive can be a user friendly adhesive that maintains contact with skin. In particular, in between the first crease 150 and the second crease 160, an adhesive can be inserted on a bottom side, wherein the bottom side of the shield 110 is the side that will contact the wearer once the prophylactic device 100 with the shield 110 is worn (e.g., put on). It is to be appreciated that any suitable adhesive that maintains contact with skin can be utilized such as but not limited to natural rubbers, latex, synthetic elastometric modifying agents, and/or a combination thereof. Moreover, it is to be appreciated that an adhesive that minimizes pain upon removal can also be implemented.

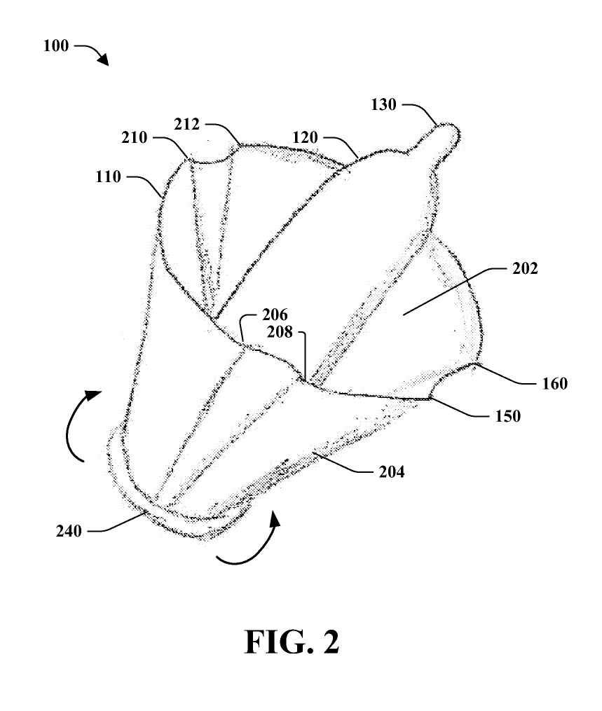

The prophylactic device 100 with the shield 110 is constructed from a single continuous piece of material. For instance, after construction of the prophylactic device 100 with the shield 110 (e.g., discussed in more detail below) utilizing molding techniques, the shield 110 is folded upward onto the shaft 120. Once folded, the creases (e.g., the first crease 150, the second crease 160, and others (if applicable)) can be utilized to ensure proper collapse or fold on the shaft 120. A bottom portion (e.g., opposite end of the end with the reservoir tip 130) on the condom portion and a top portion on the shield portion can a transition location in which both (e.g., portion of shaft and portion of shield) can be rolled to create the ring (not shown but discussed in FIG. 2). In particular, in the continuous piece of material that forms the prophylactic device 100 and the shield 110, an area where the prophylactic device 100 meets the shield 110 is folded such that the shield can be folded upwards onto the shaft. This fold can then be rolled upward to create a ring (not shown but discussed in FIG. 2). It is to be appreciated that during use, the shield 110 can be folded downward over the ring (not shown) without unrolling the entire prophylactic device 100 with shield 110.

For clarity, a crease or fold can be fabricated onto the shield 110 which allows the shield 110 to collapse onto the shaft 120 or away from the shaft 120. Such creases or folds can be similar to the first crease 150 and the second crease 160. A fold (not shown) can further exist in an area where the prophylactic device 100 meets the shield 110 to allow the shield 110 to fold upwards to the shaft 120 or downwards away from the shaft 120. This fold (not shown) can be then rolled upwards to create a ring (discussed in FIG. 2).

It is to be appreciated that the prophylactic device 100 with the shield 110 can be constructed from any suitable material such as, but not limited to, latex, non-latex, polyurethane, polyisoprene, AT-10 resin, lambskin, sheep intestine, among others. In addition, it is to be appreciated that the subject innovation is intended to include any lubricants (e.g., oil based, non-oil based, etc.) and/or spermicidal chemical (e.g., nonoxynol-9, among others) that can be coated or inserted onto or into the condom and integrated shield. Furthermore, the prophylactic device 100 and the shield 110 can be textured to include, but not limited to, ribbed, studded, among others such that the texture can be on an inside and/or an outside of the prophylactic device 100 with the shield 110. Additionally, the prophylactic device 100 with the shield 110 can come in various sizes to accommodate any wearer, wherein the size can be varying for the condom section as well as the size of the shield section.

It is to be appreciated that the prophylactic device 100 with shield 110 can include a two-material design in which the condom section (e.g., shaft 120, reservoir tip 130) can be a first material and the shield section (e.g., shield 110) can be a second material. For instance, the second material can be a stronger or more durable material in comparison to the first material. In general, the second material can be more rigid due to the tension and pressure endured versus the sensitivity required for the condom section. It is to be appreciated that the shield 110 can be stronger and more durable by applying additional coatings of a material of which both the condom 100 and the integrated shield 110 are comprised. In an embodiment, the second material is thicker than the first material. In an embodiment, the first material is a higher elasticity than the second material.

FIG. 2 illustrates a prophylactic device 100 that includes a shield that collapses upon the shaft 120 or collapses away from the shaft 120. The prophylactic device 100 is illustrated in an intermediate position between an open condition and a closed condition, wherein the open condition is collapsing the shield 110 away from the shaft 120 and a closed condition is collapsing the shield 110 toward the shaft 120. The prophylactic device 100 includes a top side 202 and a bottom side 204, wherein the top side 202 can be in contact with the shaft 120 (with a collapse of the shield 110). The prophylactic device 100 can include pairs of creases. As discussed above, the prophylactic device 100 can include at least one crease. For instance, the prophylactic device 100 can include a first pair (e.g., the first crease 150, the second crease 160), a second pair (e.g., a third crease 206, a fourth crease 208), a third pair (e.g., a fifth crease 210, a sixth crease 212), and a fourth pair (not shown but can include a seventh crease and an eighth crease), wherein each pair of creases are equidistance across the shield 110 and from one another.

In an embodiment, the creases (e.g., at least the first crease 150 and the second crease 160) are used to collapse the shield 110 to reduce surface area of the shield 110 which enables the shield 110 to lie and contact the shaft 120. In particular, the creases and folding of the shield 110 on the creases enable the outer circumference (discussed in FIG. 1) of the shield 110 to be reduced to the inner circumference (discussed in FIG. 1) of the shield 110 or the circumference of the shaft 120. At the transition location, a ring 240 can be a pivot point in which the shield 110 is collapsed upwards toward the shaft 120 (depicted with arrows) or collapsed downwards (not depicted) toward the shaft 120. This transition location can be a pivot point in which shield 110 is folded to create a fold that is rolled to create the ring 240.

To utilize the prophylactic device 100 with the shield 110, the ring 240 can be rolled downward to expose the shaft 120 with the shield 110 collapsed thereupon. Based upon a distance the ring 240 is rolled downward, a larger amount of shaft 120 is exposed as well as a larger amount of shield 110. It is to be appreciated that the size of the shield 110 and the prophylactic device 100 can be adjusted for any suitable size. For instance, a large size can include a larger distance for unrolling to expose the shield 110 (e.g., the shaft 120 is a longer). In another example, a small size can include an overlap of the shield 110 passed the shaft 120 (e.g., the shaft is shorter)

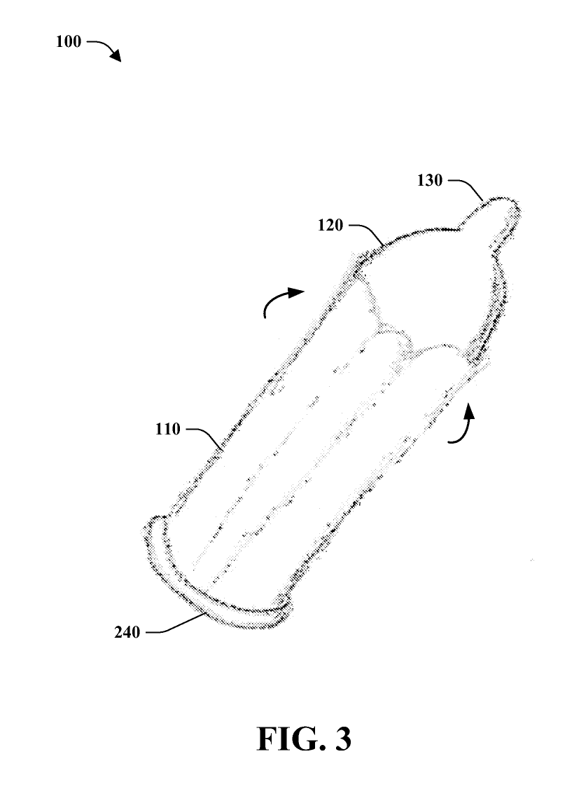

FIG. 3 illustrates the prophylactic device 100 with a shield folded onto the shaft 120. The device 100 is shown with the shield 110 collapsed upon the shaft 120 such that the top side (not shown but discussed in FIG. 2) of the shield 110 is in contact with the shaft 120 and a portion of the bottom side (not reference but discussed in FIG. 2) is opposite the top side. It is to be appreciated that a portion of the bottom side is concealed in one or more creases when the shield 110 is collapsed (illustrated with arrows) onto the shaft 120. The surface area of the shield 110 is greatly reduced due to the folding on the creases (not shown).

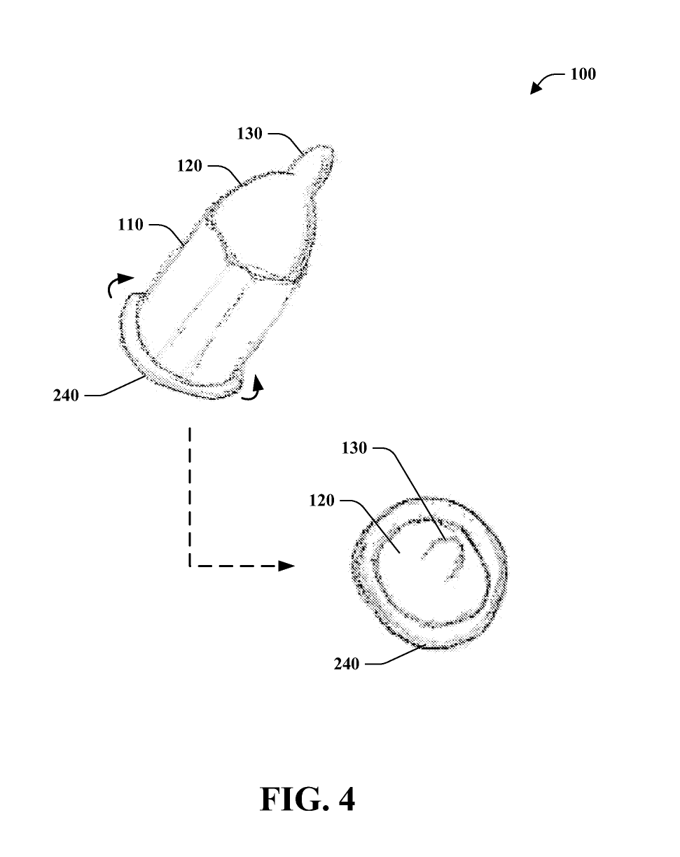

FIG. 4 illustrates the prophylactic device 100 with the shield 110 in a rolled position (closed). The device 100 is shown in a process of being rolled up (e.g., closed) from the ring 240. The ring 240 is created upon a collapse of the shield 110 upward toward the shaft 120, wherein the collapse is at a pivot point approximate to a transition location. The collapse of the shield 110 on the transition location enables a rolling or unrolling for a closed condition or an open condition respectively. The rolling allows for the device 100 to be packaged in a container or packaging of a size corresponding to a circumference of the ring 240. The arrow illustrates a completely rolled up device 100 with shield 110. It is to be appreciated that the ring 240 can include both a portion of the shaft 120 and a portion of the shield 110. The wearer can unpack the device 100 by rolling down the ring 240 until an amount of the device 100 is exposed to cover the wearer's penis (e.g., shaft of the device) and the wearer's pubic region (e.g., shield of the device). In other words, a wearer would roll down the ring 240 and follow FIG. 4, FIG. 3, FIG. 2, and lastly FIG. 1 with downward motions in which each figures corresponds to a stage of unrolling the device 100.

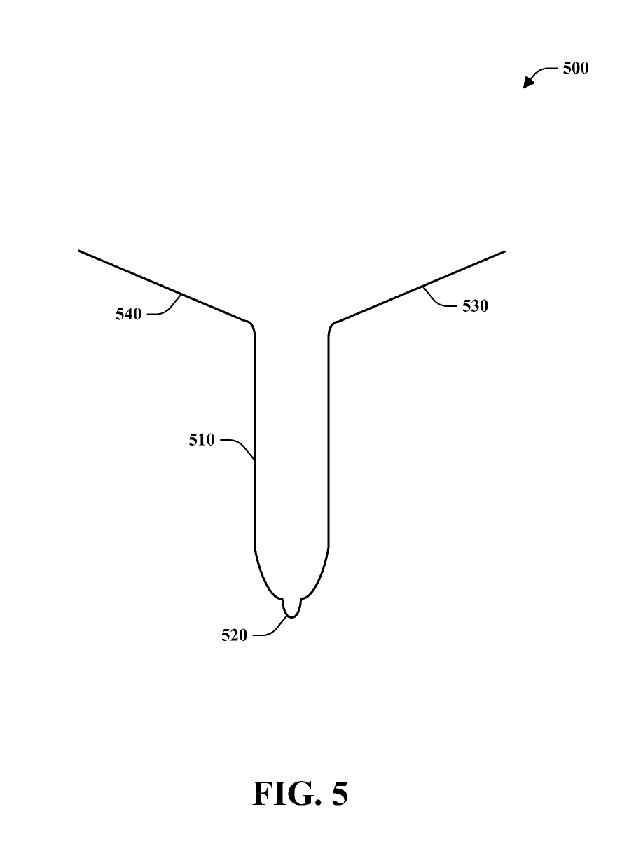

FIG. 5 is a cross-sectional view 500 of a mold for the prophylactic device in accordance with the subject matter. The cross-sectional view 500 of a mold can be made of any suitable material such as, but not limited to, glass, ceramic, among others. The cross-sectional view 500 can include a shaft region 510 on the mold to accommodate the shaft of a wearer's penis. It is to be appreciated that the circumference and length of the shaft can be any suitable size in order to fit a penis. The cross-sectional view 500 of the mold can include a reservoir tip 520. The reservoir tip 520 can be any suitable size or shape in order to capture semen from a penis. The cross-sectional view 500 of the mold can further include a right shield region 530 and a left shield region 540. The right shield region 530 and the left shield region 540 together can form a frustoconical shape with a top transitioning to the shaft region 510 and a base opposite of the top. It is to be appreciated that an angle from the shaft region 510 to either shield region (e.g., right shield region 530 or left shield region 540) can be any suitable angle to create a frustoconical shape (e.g., cone-like shape with a top cut off). Moreover, a length of either the right shield region 530 or the left shield region 540 can be any suitable length in order to provide a surface area for the shield. Moreover, the cross-sectional view 500 of the mold can include at least one or more creases (not shown) on either the right shield region 530 or the left shield region 540. In an embodiment, a ridge or crevasse can be on the shield region (e.g., right shield region 530 and/or left shield region 540) to create a fold or crease.

In an embodiment, the mold represented by the cross-sectional view 500 can be utilized in a dipping process to create a prophylactic device as disclosed herein. For instance, the mold can be dipped and subsequently dried, wherein such steps can be repeated as necessary to get a particular thickness or layers of a material that the device is made from. In another embodiment, a shield portion can be pre-fabricated and attached to the shield regions on the mold and the dipping process can be performed to integrate the shield into the shaft region 510 and reservoir region 520, wherein the integration is at a transition location in which the shaft connects and transitions to the shield.

The aforementioned devices, and the like have been described with respect to interaction between several components and/or elements. It should be appreciated that such devices and elements can include those elements or sub-elements specified therein, some of the specified elements or sub-elements, and/or additional elements. Further yet, one or more elements and/or sub-elements may be combined into a single component to provide aggregate functionality. The elements may also interact with one or more other elements not specifically described herein for the sake of brevity, but known by those of skill in the art.

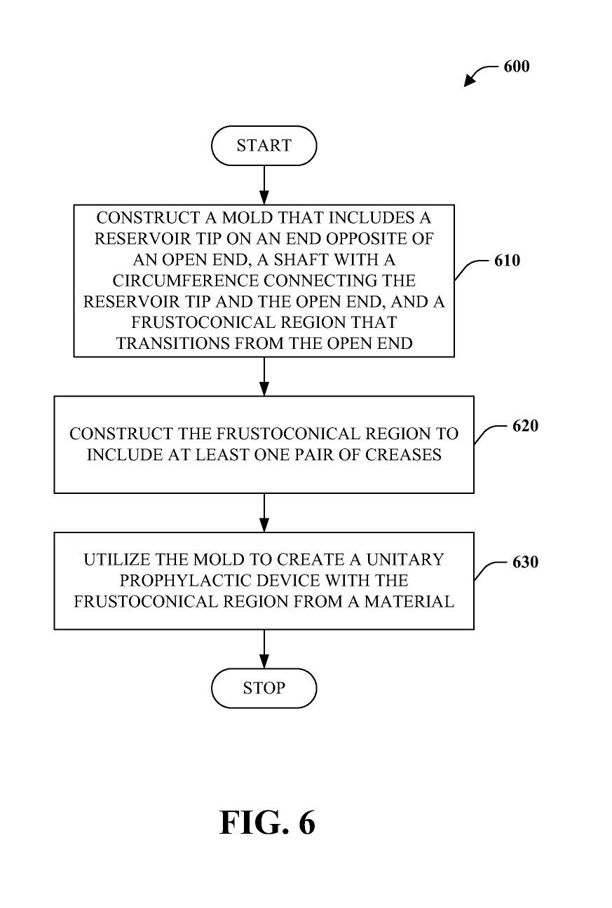

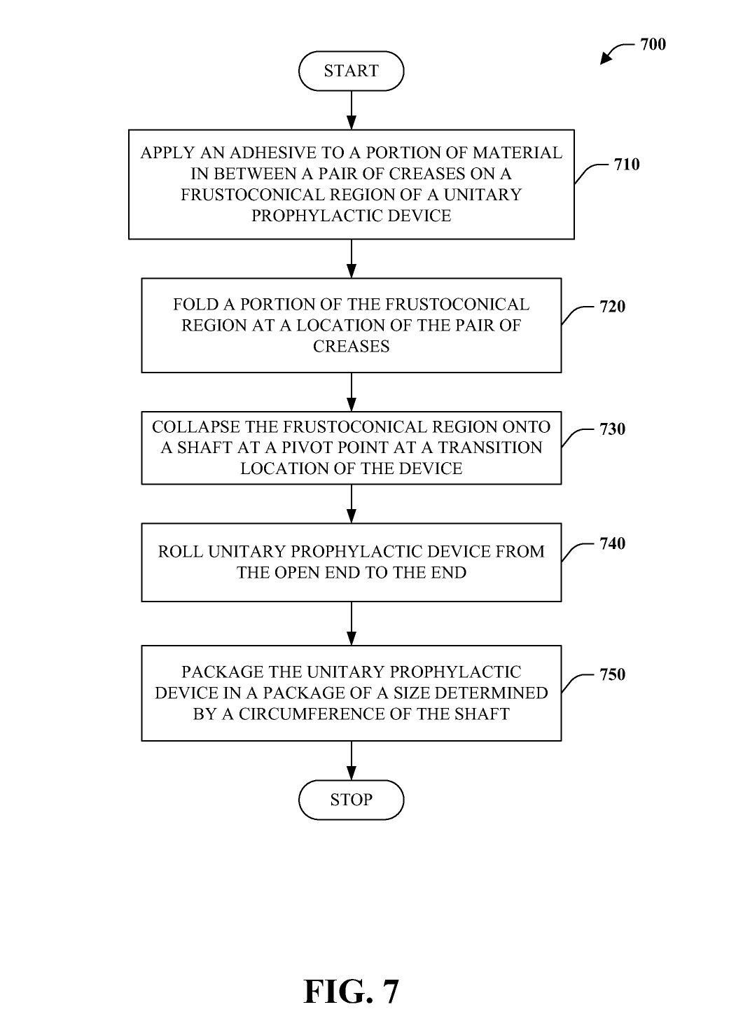

In view of the exemplary devices and elements described supra, methodologies that may be implemented in accordance with the disclosed subject matter will be better appreciated with reference to the flow charts of FIGS. 6-7. While for purposes of simplicity of explanation, the methodologies are shown and described as a series of blocks, it is to be understood and appreciated that the claimed subject matter is not limited by the order of the blocks, as some blocks may occur in different orders and/or concurrently with other blocks from what is depicted and described herein. Moreover, not all illustrated blocks may be required to implement the methods described hereinafter.

FIG. 6 is a flow chart diagram of a method 600 of creating a condom with an integrated shield. At reference numeral 610, a mold can be constructed that includes a reservoir tip on an end opposite of an open end, a shaft with a circumference connecting the reservoir tip and the open end, and a frustoconical region that transitions from the open end. In particular, a mold as illustrated in a cross-sectional view in FIG. 5 can be constructed. The mold can be constructed from any suitable material such as, but not limited to, glass, ceramic, among others.

At reference numeral 620, the frustoconical region can be constructed to include at least one pair of creases. In particular, the mold can include a ridge or crevasse to enable a crease or fold to be fabricated on the frustoconical region. At reference numeral 630, the mold can be utilized to create a unitary prophylactic device with the frustoconical region from a material. By way of example and not limitation, the mold can be dipped into a material such that the material coats the mold. Upon drying, the material can be removed from the mold to create a condom with an integrated shield from one piece of such material. It is to be appreciated that the mold can be dipped into a material two or more times and dried between each dipping to allow for a stronger construction of the device.

FIG. 7 is a flow chart diagram of a method 700 of molding a condom with an integrated shield. At reference numeral 710, an adhesive can be applied to a portion of material in between a pair of creases on a frustoconical region of a unitary prophylactic device. At reference numeral 720, a portion of the frustoconical region can be folded at a location of the pair of creases. At reference numeral 730, the frustoconical region can be collapsed onto a shaft of the device at a pivot point located at a transition location of the device. In an embodiment, the collapsing of the frustoconical region onto the shaft is at an intersection point (e.g., a location on the condom in which the shaft ends and the shield begins). The folding on the creases and the collapse onto the shaft of the condom allows for the shield to form to a circumference substantially similar to the circumference of the shaft portion of the condom, wherein such configuration facilitates rolling or unrolling the device and/or packaging thereof. In particular, the shield is integrated with the condom and the first crease and the second crease are included onto such shield.

At reference numeral 740, the unitary prophylactic device can be rolled from the open end to the end. In an embodiment, the rolling can create a ring. At reference numeral 750, the unitary prophylactic device can be packaged based upon a size determined by approximately a circumference of the shaft or the ring. In particular, the packaging can be based upon a total circumference that include the circumference of the shaft plus the ring width resultant from the device being rolled up (e.g., closed).

The unitary prophylactic device can be a circular “prophylactic dam” at the base of such condom. The prophylactic dam (also referred to as a shield) enhances protective qualities of the condom by providing an additional barrier between the wearer's pubic region and the genitals of the partner. Generally, unitary prophylactic device offers protection to the external genital region, mucus membranes, among others. Additional protection against health risks and sexually transmitted infections is afforded by the shield providing a sterile barrier against vaginal, anal, other bodily secretions, among others.

The unitary prophylactic device, as discussed above, is comprised of one continuous piece of latex. In addition to being more sanitary, it is more convenient and discrete in use, application, purchase, transport, and/or mobility. Furthermore, the unitary prophylactic device can be packaged in a compact and familiar fashion for consumers, yet traditional condoms do not include an integrated shield.

In an embodiment, a prophylactic device is provided that includes a condom section that includes a shaft, a reservoir tip, and an open end opposite of the reservoir tip, the shaft includes a circumference; a shield section transitioned from the end of the condom section that includes at least one crease, the shield section is a frustoconical shape with a base and a top and the top transitioning to the open end of the condom; the shield section includes an inner circumference at the top and an outer circumference at the base, the inner circumference is less than the circumference of the shaft of the condom section; the inner circumference is proximate to the circumference of the shaft of the condom section; and the condom section and the shield section are formed from a continuous and single piece of a material.

In an embodiment, the device can include a ring created from a portion of the open end of the condom section and a portion of the top of the shield section rolled upward on the shaft of the condom section toward the reservoir tip, wherein the ring is located at an area in which the condom section transitions to the shield section. In an embodiment, the shield section is configured to at least one of: collapse upwards onto the shaft of the condom section based upon the at least one crease or the ring; collapse downwards away from the shaft of the condom section based upon the at least one crease or the ring. In an embodiment, the material is at least one of a latex, a non-latex, a polyurethane, a polyisoprene, an AT-10 resin, a lambskin, or a sheep intestine.

In an embodiment, the device can include an adhesive applied on a bottom surface of the shield section, the bottom surface is opposing a top surface that folds onto an exterior of the shaft of the condom section. In an embodiment, the adhesive is applied between an area between two creases on the shield portion. In an embodiment, the condom section is a first thickness of the material and the shield section is a second thickness of the material. In an embodiment, the second thickness is greater than the first thickness of material.

In an embodiment, a method for creating a condom can be providing that includes constructing a mold that includes a reservoir tip on an end opposite of an open end, a shaft with a circumference connecting the reservoir tip and the open end, and a frustoconical region that transitions from the open end; constructing the furstoconical region to include at least one pair of creases; and utilizing the mold to create a unitary prophylactic device with the frustoconical region from a material. In an embodiment, the material is at least one of a latex, a non-latex, a polyurethane, a polyisoprene, an AT-10 resin, a lambskin, or a sheep intestine.

In an embodiment, the method can include applying an adhesive to a portion of the material in between the pair of creases on the frustoconical region. In an embodiment, the method can include folding a portion of the frustoconical region at a location of the pair of creases; and collapsing the frustoconical region onto the shaft at a pivot point at a transition location. In an embodiment, the method can include rolling the unitary prophylactic device from the open end to the end. In an embodiment, the method can include packaging the unitary prophylactic device with the frustoconical region in a package of a size determined by approximately a circumference of the shaft.

In an embodiment, a unitary prophylactic device can be provided that includes a condom portion that includes a first end opposite of second end, wherein the second end is a reservoir tip and the first end is connected to the reservoir tip by a shaft having a length and a circumference; a shield portion of a frustoconical shape having a top and a base, the top transitions into the first end of the condom portion, the shield portion includes an inner circumference and an outer circumference separated by a distance; the inner circumference is approximate to the circumference of the shaft of the condom portion; the shield portion includes the following: a first crease and a second crease that each extend from the inner circumference and the outer circumference for the distance, wherein the first crease and the second crease are a first pair of creases; a third crease and a fourth crease that each extend from the inner circumference and the outer circumference for the distance, wherein the third crease and the fourth crease are a second pair of creases; a fifth crease and a sixth crease that each extend from the inner circumference and the outer circumference for the distance, wherein the fifth crease and the sixth crease are a third pair of creases; and a seventh crease and an eighth crease that each extend from the inner circumference and the outer circumference for the distance, wherein the seventh crease and the eighth crease are a fourth pair of creases; and the first pair, the second pair, the third pair, and the fourth pair of creases are equidistance from one another.

In an embodiment, the condom portion and the shield portion are made from at least one of a latex, a non-latex, a polyurethane, a polyisoprene, an AT-10 resin, a lambskin, or a sheep intestine. In an embodiment, the unitary prophylactic device can include an adhesive material on a bottom side of the shield portion, the bottom side is opposing a top side that folds onto an exterior of the shaft of the condom portion. In an embodiment, the unitary prophylactic device can include a portion of adhesive located on at least one of the following: an area on the a bottom side of the portion of shield between the first crease and the second crease; an area on the a bottom side of the portion of shield between the third crease and the fourth crease; an area on the a bottom side of the portion of shield between the fifth crease and the sixth crease; and an area on the a bottom side of the portion of shield between the seventh crease and the eighth crease.

In an embodiment, the unitary prophylactic device can include a first thickness for the condom portion and a second thickness for the shield portion, the first thickness is less than the second thickness. In an embodiment, the unitary prophylactic device can include a first elasticity for the condom portion and a second elasticity for the shield portion, the first elasticity is higher than the second elasticity.

The figures above sequences, among others, the actual application of the condom with integrated shield in reverse. Such figures can be utilized to illustrate packaging options. The structural ribs (also referred to as creases or folds) described above can be elastic in nature. Therefore, such configuration (e.g., shield open toward the pubic region of the wearer) can be considered the “resting” position of the condom with integrated shield. Furthermore, when the condom is open, compression of the folded shield can be released, allowing the shield to return to the open position (e.g., resting position) while the structure of the shield can be maintained by the creases and/or folds (e.g., also referred to as ribs).

What has been described above includes examples of the subject innovation. It is, of course, not possible to describe every conceivable combination of components or methodologies for purposes of describing the claimed subject matter, but one of ordinary skill in the art may recognize that many further combinations and permutations of the subject innovation are possible. Accordingly, the claimed subject matter is intended to embrace all such alterations, modifications, and variations that fall within the spirit and scope of the appended claims.

Specific embodiments of an innovation are disclosed herein. One of ordinary skill in the art will readily recognize that the innovation may have other applications in other environments. In fact, many embodiments and implementations are possible. The following claims are in no way intended to limit the scope of the subject innovation to the specific embodiments described above. In addition, any recitation of “means for” is intended to evoke a means-plus-function reading of an element and a claim, whereas, any elements that do not specifically use the recitation “means for”, are not intended to be read as means-plus-function elements, even if the claim otherwise includes the word “means”.

The aforementioned systems have been described with respect to interaction between several components. It can be appreciated that such systems and components can include those components or specified sub-components, some of the specified components or sub-components, and/or additional components, and according to various permutations and combinations of the foregoing. Sub-components can also be implemented as components communicatively coupled to other components rather than included within parent components (hierarchical). Additionally, it should be noted that one or more components may be combined into a single component providing aggregate functionality or divided into several separate sub-components, and any one or more middle layers, such as a management layer, may be provided to communicatively couple to such sub-components in order to provide integrated functionality. Any components described herein may also interact with one or more other components not specifically described herein but generally known by those of skill in the art.

Although the subject innovation has been shown and described with respect to a certain preferred embodiment or embodiments, it is obvious that equivalent alterations and modifications will occur to others skilled in the art upon the reading and understanding of this specification and the annexed drawings. In particular regard to the various functions performed by the above described elements (e.g., components, assemblies, devices, compositions, garments, materials, etc.), the terms (including a reference to a “means”) used to describe such elements are intended to correspond, unless otherwise indicated, to any element which performs the specified function of the described element (e.g., that is functionally equivalent), even though not structurally equivalent to the disclosed structure which performs the function in the herein illustrated exemplary embodiment or embodiments of the innovation. In addition, while a particular feature of the innovation may have been described above with respect to only one or more of several illustrated embodiments, such feature may be combined with one or more other features of the other embodiments, as may be desired and advantageous for any given or particular application. Although certain embodiments have been shown and described, it is understood that equivalents and modifications falling within the scope of the appended claims will occur to others who are skilled in the art upon the reading and understanding of this specification.

In addition, while a particular feature of the subject innovation may have been disclosed with respect to only one of several implementations, such feature may be combined with one or more other features of the other implementations as may be desired and advantageous for any given or particular application. Furthermore, to the extent that the terms “includes,” “including,” “has,” “contains,” variants thereof, and other similar words are used in either the detailed description or the claims, these terms are intended to be inclusive in a manner similar to the term “comprising” as an open transition word without precluding any additional or other elements.

What is claimed is:

1. A prophylactic device, comprising:
a condom section that includes a shaft, a reservoir tip, and an open end opposite of the reservoir tip, the shaft includes a circumference;
a shield section transitioned from the end of the condom section that includes at least one crease, the shield section is a frustoconical shape with a base and a top and the top transitioning to the open end of the condom;
the shield section includes an inner circumference at the top and an outer circumference at the base, the inner circumference is less than the circumference of the shaft of the condom section;
the inner circumference is proximate to the circumference of the shaft of the condom section; and
the condom section and the shield section are formed from a continuous and single piece of a material.

2. The prophylactic device of claim 1 further comprises a ring created from a portion of the open end of the condom section and a portion of the top of the shield section rolled upward on the shaft of the condom section toward the reservoir tip, wherein the ring is located at an area in which the condom section transitions to the shield section.

3. The prophylactic device of claim 2, the shield section is configured to at least one of:
collapse upwards onto the shaft of the condom section based upon the at least one crease or the ring; and
collapse downwards away from the shaft of the condom section based upon the at least one crease or the ring.

4. The prophylactic device of claim 1, the material is at least one of a latex, a non-latex, a polyurethane, a polyisoprene, an AT-10 resin, a lambskin, or a sheep intestine.

5. The prophylactic device of claim 1 further comprises an adhesive applied on a bottom surface of the shield section, the bottom surface is opposing a top surface that folds onto an exterior of the shaft of the condom section.

6. The prophylactic device of claim 5, the adhesive is applied between an area between two creases on the shield portion.

7. The prophylactic device of claim 1, the condom section is a first thickness of the material and the shield section is a second thickness of the material.

8. The prophylactic device of claim 7, the second thickness is greater than the first thickness of material.

9. A method of creating a prophylactic device, comprising:
constructing a mold that includes a reservoir tip on an end opposite of an open end, a shaft with a circumference connecting the reservoir tip and the open end, and a frustoconical region that transitions from the open end;
constructing the frustoconical region to include at least one pair of creases; and
utilizing the mold to create a unitary prophylactic device with the frustoconical region from a material.

10. The method of claim 9, the material is at least one of a latex, a non-latex, a polyurethane, a polyisoprene, an AT-10 resin, a lambskin, or a sheep intestine.

11. The method of claim 9, further comprising applying an adhesive to a portion of the material in between the pair of creases on the frustoconical region.

12. The method of claim 9, further comprising:
folding a portion of the frustoconical region at a location of the pair of creases; and
collapsing the frustoconical region onto the shaft at a pivot point at a transition location.

13. The method of claim 12, further comprising rolling the unitary prophylactic device from the open end to the end.

14. The method of claim 13, further comprising packaging the unitary prophylactic device with the frustoconical region in a package of a size determined by approximately a circumference of the shaft.

15. A unitary prophylactic device, comprising:
a condom portion that includes a first end opposite of second end, wherein the second end is a reservoir tip and the first end is connected to the reservoir tip by a shaft having a length and a circumference;
a shield portion of a frustoconical shape having a top and a base, the top transitions into the first end of the condom portion, the shield portion includes an inner circumference and an outer circumference separated by a distance;
the inner circumference is approximate to the circumference of the shaft of the condom portion;
the shield portion includes the following:
a first crease and a second crease that each extend from the inner circumference and the outer circumference for the distance, wherein the first crease and the second crease are a first pair of creases;
a third crease and a fourth crease that each extend from the inner circumference and the outer circumference for the distance, wherein the third crease and the fourth crease are a second pair of creases;
a fifth crease and a sixth crease that each extend from the inner circumference and the outer circumference for the distance, wherein the fifth crease and the sixth crease are a third pair of creases; and
a seventh crease and an eighth crease that each extend from the inner circumference and the outer circumference for the distance, wherein the seventh crease and the eighth crease are a fourth pair of creases; and
the first pair, the second pair, the third pair, and the fourth pair of creases are equidistance from one another.

16. The unitary prophylactic device of claim 15 further comprises the condom portion and the shield portion are made from at least one of a latex, a non-latex, a polyurethane, a polyisoprene, an AT-10 resin, a lambskin, or a sheep intestine.

17. The unitary prophylactic device of claim 15, further comprising an adhesive material on a bottom side of the shield portion, the bottom side is opposing a top side that folds onto an exterior of the shaft of the condom portion.

18. The unitary prophylactic device of claim 15, further comprising a portion of adhesive located on at least one of the following:
an area on the a bottom side of the portion of shield between the first crease and the second crease;
an area on the a bottom side of the portion of shield between the third crease and the fourth crease;
an area on the a bottom side of the portion of shield between the fifth crease and the sixth crease; and
an area on the a bottom side of the portion of shield between the seventh crease and the eighth crease.

19. The unitary prophylactic device of claim 15, further comprising a first thickness for the condom portion and a second thickness for the shield portion, the first thickness is less than the second thickness.

20. The unitary prophylactic device of claim 15, further comprising a first elasticity for the condom portion and a second elasticity for the shield portion, the first elasticity is higher than the second elasticity.
